(19) United States
(12) Patent Application Publication
(10) Pub. No.: US 2003/0208243 A1
(43) Pub. Date: Nov. 6, 2003

(54) Inflectional therapy for mental depression and anxiety

(76) Inventor: Daniel L. Cobble, Louisville, KY (US)

Correspondence Address: Daniel L. Cobble, Ste. 12, 3401 Lesway Ct., Louisville, KY, 40220, US

(21) Appl. No.: 10/136,167

(22) Filed: May 1, 2002

(51) Int. Cl. A61N 1/18

(52) U.S. Cl. 607/45

(57) ABSTRACT

A therapeutic process, called inflectional therapy, is essentially a single-person process for diminishing the mental ailments of depression and anxiety. As the sufferer, being said single person, experiences such an ailment, [she]/he commences to record his thoughts and feelings by verbal expression onto a recording medium, such as an audio tape recorder. This is called a session, and the ensuing recording is called a recorded session. Typically, multiple sessions comprise adequate therapy for depression, but a single session usually diminishes a bout of anxiety. In circumstances where the sufferer does not have ready access to his recorder at the time of the ailment, he may commence recording his thoughts and feelings of the “prior” ailment when he eventually has access. Also, the sufferer may include positive, verbal expressions of encouragement during a session. A person may even record those thoughts and feelings when he is not undergoing a current ailment, such as inflecting upon happiness and other positive thoughts, or aspects of a personality disorder. Then, as desired, he listens to the recorded sessions for examining his ailment from the objective, recorded format, for the self-observation that leads to self-objectivity. But more significant, therapeutic value lies in the act(s) of expressing one's thoughts and feelings within the session(s) through the mental “focal point” of the recording medium.—For these matters, inflectional therapy may be utilized in institutional environments, such as hospitals, businesses, the military, etc., to treat patients, and to calm personnel during situations of conflict.

### BACKGROUND OF THE INVENTION

The present invention comprises a process of “self-therapy” for diminishing mental depression and anxiety. For this specification, mental depression is defined as debilitating psychological orientation, trauma, and mental abuse that leads respectively to feelings of low self-esteem, disillusionment, fear, and feelings of inadequacy. The present invention may also be appropriate for conditions of mental depression as may be caused by chemical imbalance or bi-polar disorders.

Also for this specification, anxiety is defined as varying degrees of emotional disturbance arising from one's environment, or, one's internally mental tension brought on by stress. Causes of anxiety include insomnia, despair, grief, distress, sadness, anguish, anger, fear, rejection, embarrassment, and worry.

For centuries, sufferers of depression and anxiety have agonized over the problems of receiving adequate therapy that would provide suitable relief Of course, the ideal situation would be a cure.

For depression, there are several theories that attempts to explain its causes, as mental health experts have striven to find suitable treatments, especially for acute conditions. Through the twentieth century, psychologists and psychiatrists have devised and re-devised various therapies. Many depression sufferers having regular access to the “traditional” counseling-type of therapies sometimes acquire a chance of prevailing against depression. This is especially true for those sufferers having more prosperous livelihoods who can afford to pay for these treatments. By contrast, low income sufferers in very many cases go untreated.

By the late twentieth century anti-depressant drugs have evolved in the marketplace. Many of these very sophisticated medicines can provide sustained relief in many situations of chemically-based depression. However, these drugs have various problematic side effects, in which some may be very serious. And, often times, they are not adequate in matters of psychological depression.

For anxiety and anxiety attacks, the environmental consequences are well known to most everyone. Cerebral tension from anxiety can intensify, which can manifest physical aches and pains, headaches, and tightness in muscles and joints. If a treatment process can impact the originating mental cause of anxiety, then these incapacitive outcomes may be avoided.

Of course, a therapy of value for anxiety must, by its very nature, promptly neutralize these effects. And, for mental depression, the therapy would achieve positive effects over time. Yet, both ailments consist of mental resistance to storing away excessively troubling emotionally-based thoughts that come from undesirable experiences. This “natural process” of memory storage is called, “memory filing.” Depressive thoughts involve unwanted memory “recall,” and anxiety and anxiety attacks involve memory generation that come from undesirable or unfortunate experiences.

So, ultimately, what is needed is a standardized as well as pervasively workable therapy for mental depression and anxiety that brings about real relief for sufferers. Even further, if this therapeutic process could be inexpensive, then most sufferers would be able to afford the opportunity to recover from both of these debilitating maladies.

### SUMMARY OF THE INVENTION

The present invention, being a new therapeutic process, is called “An Inflectional Therapy for Mental Depression and Anxiety.” This therapy is, essentially, a single person process of “self-therapy” for diminishing mental depression and anxiety, including manic depression and anxiety attacks. Within this specification, the terms depression and anxiety, and their derivative terms, are used interchangeably for text simplification, since inflectional therapy affect both of these ailments in the same way.

The term “inflect,” being the root term of inflectional, implies that the sufferer reflects “inward” within herself/himself, and for this process, he privately does so while actually experiencing a mental ailment. No mental health practitioner or medication is needed during the actual process of inflection. And, the sufferer may conduct this therapy practically anywhere, anytime, and thereby, it is assessable to almost anyone, when needed. Wherefore, inflectional therapy is inexpensive, as only an electronic recording device is needed to record “sessions” in a process of verbal inflection.

Accordingly, various rules apply for achieving effectual inflectional therapy. Generally, these rules involve the recording and listening phases; but other details apply, as also described, below.

In the recording phase, while experiencing a bout of depression, the sufferer proceeds to verbally express his thoughts, feelings and emotions onto a recording medium as he is suffering. This is called a session, and the ensuing recording is called a recorded session. A choice of recording mediums include, but not limited to, audio tape recording, video recording with audio recording, audio recording by electronic digitalization, and recording by writing. A session would continue as the depression continues; 10, 20 or 30 minutes, for instance, or even hours into a “current” state of depression. The sufferer speaks of his thoughts, feelings and emotions as he is experiencing them.

However, upon the time of depression or anxiety that he does not have immediate access to a recorder, he would verbally record/express his mentally depressed experience at a later, available opportunity. In such case, he would summarize by recording what he had felt earlier.

Typically, inflectional therapy involve multiple sessions over time for bringing recurring bouts of depression into remission. This process functions to progressively diminish succeeding bouts with less and less frequency and respectively regressive emotional intensity. Of course, the frequency of sessions would depend on the nature and severity of the sufferer's condition. Still, the success of inflectional therapy requires the willingness of the sufferer to “step up” to reveal himself to himself. Yet, even this prospect is made much easier, since a session is performed privately with the single participant in inherent control.

For anxiety, a session is conducted as needed for any given period of angst. It has even proven to be effective for diminishing anger and sleep insomnia. Here, inflection usually results in the prompt subsiding of the condition within a single session.

If the recording medium's recording unit, such as an audio cassette tape, is not full after completing a session, the sufferer simply stops the recording. Then, when he is ready to commence recording again for continuing the same or “next” session, he starts recording from the end location of the prior recorded session. He would continue successive recordings over time as he experiences depression, until the tape is full. Next, he would begin the listening phase, by listening to the series of recorded sessions as needed. Of course, he may listen to a recorded session at anytime.

While recording a session, the point-in-time-information of when the session is recorded may be recorded with a session. A choice of data for this information includes a day of the week, a calendar date, and a time of day. One or more choices of such data as desired may be recorded onto the recorded session. Accordingly, the sufferer may record the day, date and time onto the recording before he commences to speak about his ailment.

The purpose of date-recorded sessions is to provide the sufferer or other person with a reference point of the progression of successive sessions. Here, the sufferer more easily correlates the sequence of listening to recorded sessions, especially when he is preoccupied with other activities while listening.

As a matter of course, a primary function of these “specialized” recordings is that they relegate the sufferer to focus his debilitating thoughts and feelings, his anxiety, onto the exacting recording medium. Since the sufferer records his thoughts and feelings during the ailment, he is in direct contact with himself as he is suffering, and thus, his inflection guides him to face his feelings with a high degree of mental focus, the inflection being directed succinctly onto the recorder. By expressly verbalizing these feelings, he gives them full attention; he privately and fully “vents” his anxiety, which serves to silence his mind. To put another way, with no further mental resistance, his troubling thoughts are “filed away,” given “full” and “due” conscious observation and attention. Hence, natural memory filing is induced by inflectional thereapy.

Consequently, it does not matter whether the sufferer is ailing from mental depression or anxiety.

Therefore, these recordings are typically very detailed and personal in nature, and the sufferer may not wish to share them with anyone. Even if he is being assisted by a therapist or other practitioner, it should be his decision as to whether he wishes to disclose his recorded sessions.

This process is in stark contrast to traditional therapy that comprises the patient being in session with a psychoanalytical practitioner (psychologist, psychiatrist, etc), therapist, or counselor. In such cases, the patient shares his “past” experiences with the practitioner, whether short-term or long-term. Then the practitioner makes her/his psychological assessment in accordance with the patient's memory and mental disposition. Also, the patient may establish and/or interpret his own perspectives through this personal contact with the practitioner. Therefore, traditional therapy is necessarily a two-person process in which the practitioner is in control, and therefore must necessarily “effect” the therapeutic process through his participation. Of course, these traditional therapies are designed that way.

For inflectional therapy, however, the sufferer maintains innate control, and proceeds comfortably at his own pace and manner, even if he is otherwise being advised by a practitioner. For example, the therapist may show or practice with the sufferer on how to perform a session. The therapist may also act as a guide, and even monitor the patient's progress. However, the therapist typically remains “outside” the functioning realm of the process, unless the patient approves of the therapist actually witnessing a session or later listening to his recorded session. This is because the process is a means of focused, personal inflection through verbalized self-expression, as prompted by the act of recording. Therefore, professional evaluation should only be conducted when the patient understands and approves of this step. Also, no such personnel file on the sufferer regarding this therapy should be kept, unless otherwise sought and/or agreed upon.

If the sufferer has a fundamental or technical understanding of his condition, then he may add positive, beneficial, and therapeutic type messages to the session. Accordingly, he may state positively encouraging statements after describing a low, depressive state of mind. For example, “I must not blame myself for this problem. This is not my fault.” Or, he may iterate a technical description of what he is feeling, such as clinically identifying his condition. For example, he may relate his current condition to his troubled childhood.

Moreover, for those sufferers who do not understand their condition should seek professional help for this purpose. Inflectional therapy becomes woefully more effective when the sufferer can identify his condition and verbally iterate such information in his sessions. Here again, the professional therapist/ practitioner can be of assistance.

Nevertheless, even if the sufferer may not be aware of his specific psychological malady, to inflect by recording ones thoughts and feelings while he is troubled is the core function of inflectional therapy.

Still yet, another aspect of the recording phase involve situations whereas the sufferer may experience negative or debilitating thoughts or feelings, being subliminal or otherwise, even though he may not be ailing at that time. Here, too, he would conduct the process of inflection by recording these thoughts and feelings by verbal expression. This inflection may also apply to cerebral and emotional reflections arising from a person's personality disorder. Again, these verbalizations may often times be more personal than those expressed in traditional therapy, because the person may and should reveal to himself his most private issues about himself, other people, and his life's situations. Thus, as already explained, he may want to restrict his recordings to only his own listening, and not share them with anyone else.

Also, a person may record those personal thoughts and “nuances” that generally tends to affect him; for example, feelings of happiness, an affinity for a co-worker, or his dislike for his boss or a neighbor. Here again, at this time, he may or may not be suffering from depressive or negative thoughts, but focus inflectional therapy on other aspects of his life, for clearly “exposing himself.”

Thereto, as a matter of practicality and productivity, the acts of stating positive, reinforcing statements of insight and understanding in the sessions will help to offset the negative and painful impact of the ailment. Constructive, verbal reinforcements of understanding should always be a part of this self-therapeutic process. The sufferer may even be creative by playing his favorite music or other peripheral sound in the audio background of the recording. So that during listening, the recordings may be even more enjoyable, especially during long periods. Some peripheral sounds may even soothe the sufferer, like the sounds of ocean waves. Additionally, affirmative, optimistic verbiage during inflection plays an important role while listening.

Personal playback of the recordings is the listening phase. Here, the sufferer may listen to the recorded sessions at any time during or after his mentally depressive experience, as desired But ideally, he would listen to a recording unit after it is filled with successive recorded sessions. This avoids the hassle of repositioning the recording unit at the end of the prior recorded session each time he begins another session. To assist even further, he may even “dedicate” a recorder only for recording, and listen on another playback system. That way, this dedicated recorder is always ready for recording the next session.

He should begin therapy with at least two recording units, as again, such as audio cassette tapes. A “writable” digital compact disc is another recording unit. In this way, he records successive sessions, from respective bouts of depression, onto the current recording unit, while in the interim he listens to prior recorded sessions from the other recording unit, at his leisure.

Typically, the sufferer would alternate these multiple recording units between “filling-up” with successive sessions and the listening phase. He records new sessions over older recorded sessions on a recording unit after another recording unit has been completed with the most recently recorded sessions. In this manner, sessions are carried out most economically by minimizing the purchasing of new recording units. This alternation of recording unit is conducive, since inflectional therapy is an informal and personal process; record keeping is not necessary. Also, audio cassette tapes are probably the most practical recording units, since cassette players are the most common playback devices in our human environments, as the cassette tape may be transported more easily within and between environments than other recording units. Of course, this alternation may be conducted with any other recording unit types, such as compact discs.

Although conducting the sessions are inherently and inordinately therapeutic, being the acts of private, emotional self-expression, the listening phase serves the important function of promoting self-observation and self-objectivity. The sufferer may relate to his depressive experience, either while he is still in the ailing state of mind, or when he is not in such state. Although listening is not required, he can listen at any time, such as while riding in his car, working on his computer, etc., to reflect and contemplate over his mental condition. By having his experiences and feelings “transferred” to the precise and focused recording format, his full objectivity may progressively become fulfilled by the listening process, for closely observing himself from outside of himself, such as a therapist does. The act of recording is the focal point for releasing his “pent up” mental energy. As self-objectivity progresses, he begins to realize that his condition is only a part, if only a small part, of his whole life. Then, his condition can no longer dominate his life, as his mental debilitation is converted to a foundation of strength.

As stated, listening is also a primary function for the affirmative, hopeful and reassuring messages that should be included in the recording of the ailment, or other inflectional circumstances, as described. In listening to his recorded sessions with reaffirming messages, the sufferer may relate to his ailment/circumstances as a condition that can be overcomed. Moreover, these positive messages, when heard repeatedly over time, can serve to reach the sufferer subliminally for prompting higher self-esteem, self-confidence, and overall improvement of personal outlook. Hence, inflectional therapy can be a true art form.

Here again, professional practitioners, such as psychologists and other therapists, can help the sufferer in formulating these positive messages and images. Also, as a “coach,” for example, the therapist may ask, “Are you conducting your sessions as you experience your emotional lows?”

Thereby, inflectional therapy may be taught and practiced in any institution. These include but are not limited to: Businesses, to increase work place production when employee depression, conflicts or anxieties arise; Schools and colleges, as a course of study for students and treatment of workers; Governmental institutions for the purposes stated above; and military institutions for the purposes stated above. Other institutions include: lecture circuits, seminars, workshops, other work place environments, community workshops, health care environments, mental health institutions, prisons, and reformatories.

Also, inflectional therapy may be explained and taught by its description being broadcasted over television, radio stations, and even computer-based broadcasts, like the internet. Furthermore, description of this process may be published in other venues which include text and computerized publications.

When performing inflectional therapy for agents/employees within an institution, the employer/administrator/boss should communicate with the employee with compassion, upon the prospect of the employee conducting a session. When the employee experiences apparent depression or anxiety, his boss should direct him to a private area, such as a small room that would also include a recording medium, such as a cassette tape recorder. If the employee does not know the “rules” of conducting the process, then the boss instructs him, basically as described, above. In addition, it is prudent that an institution's policy mandates that a session last for a definite, specified period, so as not to be too disruptive to its operations. After the employee conducts a session in private, he then exits with the recorded cassette tape in his possession. Again, the employee would retain this recorded session, for his own playback as desired, unless otherwise agreed upon. The boss would file within the company records that the session took place, and ask the employee if he feels better, and then propose that he returns to work.

If a conflict arises between multiple employees, or, for example, a supervisor and employee, then they each may be required to conduct a session. To adequately calm everyone, these sessions should ensue before further remedial action is taken. By doing so, the participants can mediate better with calmer dispositions, and thus conserve personnel energy and company resources. For example, inflectional sessions can help an employee to come to grips with a serious emotional issue that could otherwise lead to his dismissal. Or, calming the anxiety of a worker can help make the shift run smoother for everyone else.

For institutional therapy such as in a clinic, hospital or mental health facility, the therapist would likewise provide a private area for the patient/sufferer to conduct a session. The session may be accomplished before and/or after discussion with the patient. Discussion may be in the form of briefing, for instance, in directing the patient on what may be included in a session; or subsequent feedback from the patient after a session. The therapist would typically utilize inflectional therapy for providing the best overall clinical treatment for the patient's condition. Adjusting therapeutic periods for logistics and scheduling within the facility is also a consideration for administration.

Wherefore, this extraordinary self-treatment is a progressive therapeutic process that subtlety induces the sufferers self-objectivity. By inflecting upon his feelings, thoughts and emotions as depression or anxiety arises, he allows himself to “reach out” to expose himself to himself, for clear self-observation. Consequently, these problem elements may fully surface to his consciousness, to affect avoidance or the minimizing of debilitating mental conflict. To put another way, he gives due “rise” and “front-stage” facing of these issues so that they may settle quietly in his mind. They are, in effect, filed away.

So, with the only tool that the sufferer needs for conducting this therapy being a recording medium, such as an audio cassette recorder, inflectional therapy is very inexpensive for even low-income sufferers. Nothing and no one else is needed, unless the lay person requires guidance from the therapist on how the process is practiced.

Hence, depression and anxiety may become only insignificant annoyances in people's lives.

### LIST OF ILLUSTRATIONS

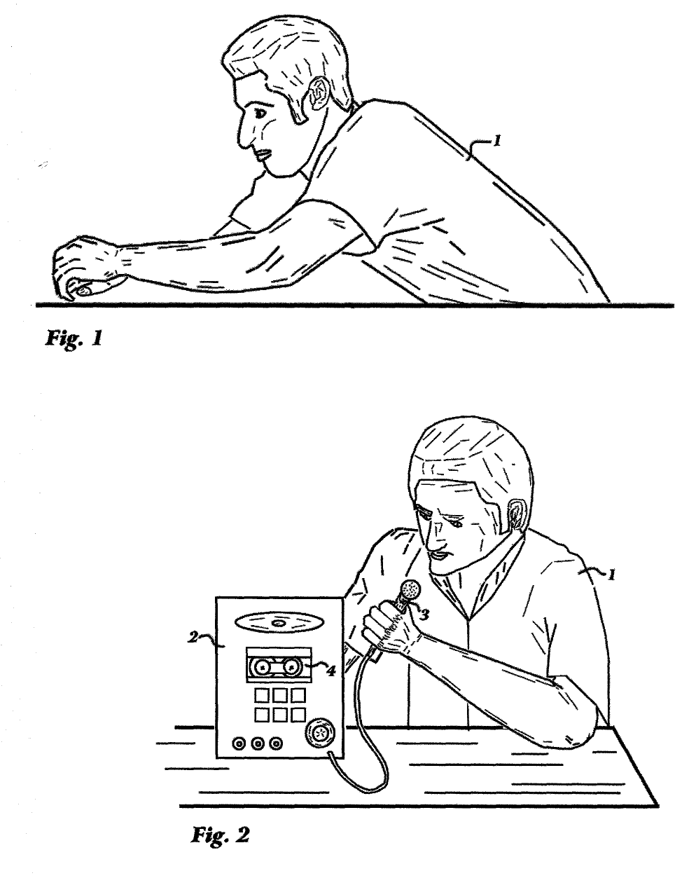

FIG. 1 shows a man in a depressed condition.

FIG. 2 shows the man conducting a session of inflectional therapy by recording his thoughts and feelings onto an audio cassette tape recorder.

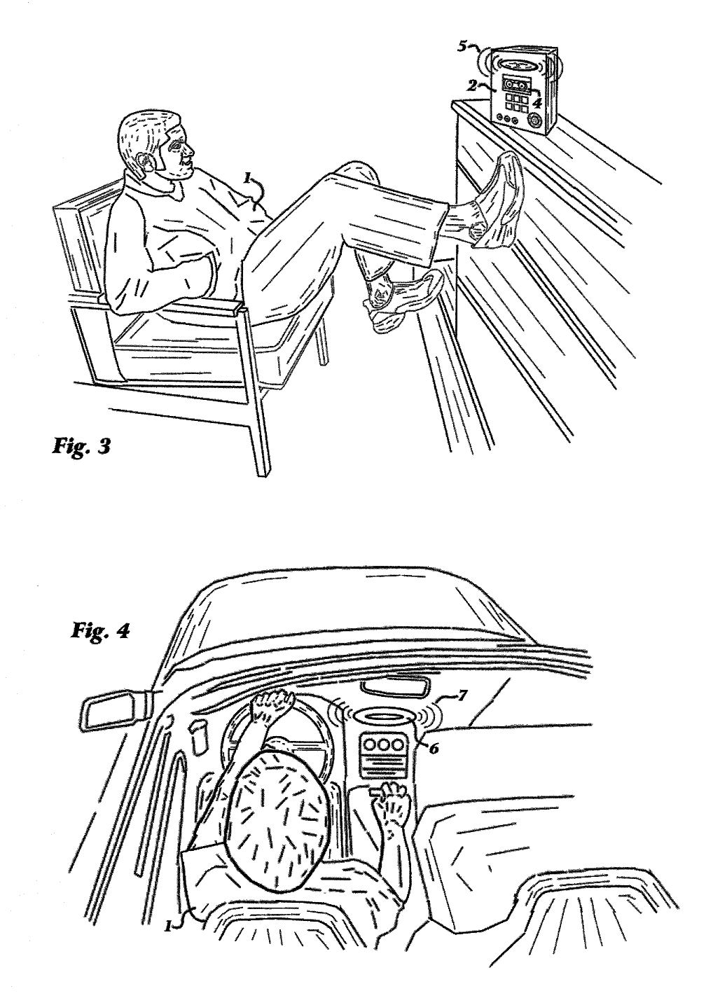

FIG. 3 shows the man conducting the listening phase of inflectional therapy by listening to his recorded sessions.

FIG. 4 shows the man listening to a recorded session in his car, while driving.

### DETAILED DESCRIPTION OF PREFERRED EMBODIMENTS

FIG. 1 shows man 1 in a mentally depressed condition, or he may be suffering from anxiety, such as anger, fear, apprehension, etc.

FIG. 2 shows man 1 conducting a session of inflectional therapy by expressing his thoughts and feelings onto audio cassette tape 4 of tape recorder 2, through microphone 3 while experiencing depression or anxiety. Or, he may be conducting a session without any ailment. The sessions are recorded as stated in the summary, above.

FIG. 2 is also applicable for an institutional setting, whereas man 1 may depict an employee or an agent of an institution being a business, government facility, etc., and is performing the recording phase of inflectional therapy in a designated private area.

FIG. 3 shows man 1 conducting the listening phase of inflectional therapy, by playing his recorded sessions on the playback function of recorder 2. Sound waves 5 depict said tape recorder playing the recorded sessions on cassette tape 4. Ideally, the cassette tape would be fully recorded on both sides with one or more sessions, as described in the summary, above. But, as also stated, filling up a recording unit is not required.

FIG. 4 shows man 1 conducting the listening phase while driving. He is listening to recorded sessions from car speaker 6 as depicted by sound waves 7. Hence, both the listening and recording phases may be conducted at virtually anyplace and anytime, for performing inflectional therapy.

What I claim is:

1. A therapeutic process, called inflectional therapy, for diminishing the mental ailments of depression and anxiety; and said process comprising a session; and said session consist of the sufferer of a ailment of said ailments recording [her]/his thoughts and feelings of said ailment by verbal expression onto a recording medium during the actual ailment and thus producing a recorded session of said session; a choice of recording mediums for said session includes audio tape recording, video recording with audio recording, audio recording by electronic digitalization, and recording by writing; and over the time of said sufferer suffering successive bouts of said ailment multiple sessions are conducted with ensuing multiple recorded sessions; and said process comprising said sufferer listening to a recorded session as needed for examining his thoughts and feelings; and said process having the therapeutic value of said sufferer expressing his thoughts and feelings in a session; and said process having the resulting therapeutic value of said sufferer externally examining his thoughts and feelings by listening to a recorded session; and an ensuing recorded session being personal of said sufferer by having recorded the details of private information of said sufferer's thoughts and feelings.

2. The therapeutic process of claim 1 including further: said process comprising said sufferer listening to a single recorded session for examining the sufferer's thoughts and feelings.

3. The therapeutic process of claim 1 including further: upon the time of said ailment that said sufferer does not have immediate access to said recording medium said sufferer records his thoughts and feelings at a later, available opportunity.

4. The therapeutic process of claim 1 including further: said sufferer will express positive and encouraging thoughts in a session.

5. The therapeutic process of claim 1 including further: a professional practitioner, including but not limited to a psychologist and a therapist, assist said sufferer in how to conduct a session.

6. The therapeutic process of claim 1 including further: a professional practitioner, including but not limited to a psychologist and a therapist, conducts said therapeutic process in an institutional environment.

7. The therapeutic process of claim 1 including further: a professional practitioner, including but not limited to a psychologist and a therapist, assists a sufferer in formulating encouragingly verbal messages for said sufferer to conduct a session.

8. The therapeutic process of claim 1 including further: a professional practitioner, including but not limited to a psychologist and a therapist, assists a sufferer in formulating verbally therapeutic messages for said sufferer to conduct a session.

9. The therapeutic process of claim 1 including further: said process is taught within a venue; said venue includes, but not limited to; the respective venues of lecture appearances, seminars, workshops, colleges and schools, businesses, work place environments, military institutions, community workshops, governmental institutions, health-care institutions, mental health institutions, prisons, and reformatories.

10. The therapeutic process of claim 1 including further: a description of said process is broadcasted by a broadcast station; and said station includes, but not limited to, respective television stations, radio stations, and computer-based broadcasts.

11. The therapeutic process of claim 1 including further: a description of said process is published in a publication; and said publication includes, but not limited to; respective text publications and computer-based publications.

12. The therapeutic process of claim 1 including further: said process is conducted in an institution; and said institution includes, but not limited to, respective lecture appearances, seminars, workshops, colleges and schools, businesses, churches, work place environments, military establishments, community workshops, governmental establishments, health-care establishments, mental health establishments, prisons, and reformatories.

13. The therapeutic process of claim 1 including further: said recording medium having a recording unit; and a choice of recording units being, but not limited to, an audio cassette tape, a writable digitized disc, and a computerized recording program; and upon said recording unit not being fully recorded after completing a recorded session, then said sufferer records a successive session onto said recording unit.

14. The therapeutic process of claim 13 including further: said sufferer alternates multiple recording units between said sessions and the listening of said recorded sessions; and said sufferer commences to record new sessions over prior recorded sessions on an existing recording unit after another recording unit has been completed with the most recent recorded session of said sufferer.

15. The therapeutic process of claim 1 including further: said recording medium having a recording unit; and a choice of recording units being, but not limited to, an audio cassette tape, a writable digitized disc, and a computerized recording program; and said sufferer plays respective peripheral sounds including, but not limited to, music and ocean waves playing in the background of said session so as to be recorded onto said recording unit of an ensuing recorded session.

16. The therapeutic process of claim 1 including further: while recording a session, point in time information is recorded onto the respective recorded session of when said session is recorded; a choice of data for said information includes a day of week, a calendar date, and a time of day.

17. A therapeutic process, called inflectional therapy, for exposing a person's thoughts and feelings to [herself]/himself, and said process comprising a session; and said session consist of the same person recording [her]/his thoughts and feelings by verbal expression onto a recording medium and thus producing a recorded session of said session; a choice of recording mediums for said session include audio tape recording, video recording with audio recording, audio recording by electronic digitalization, and recording by writing; and said person listening to said session as desired for examining his thoughts and feelings; said process having the therapeutic value of said person expressing his thoughts and feelings in a session; and said process having the resulting therapeutic value of said person externally examining his thoughts and feelings by listening to a recorded session; and the recorded session being personal of said person by having recorded the details of private information of said person 's thoughts and feelings.

18. The therapeutic process of claim 17 including further: and said process comprising multiple sessions with respectively multiple recorded sessions, and said person listening to any of said multiple sessions as desired for examining his thoughts and feelings.

19. The therapeutic process of claim 17 including further: said person conducting multiple sessions.

20. The therapeutic process of claim 17 including further: said person expresses positive and encouraging thoughts in said session.

21. The therapeutic process of claim 17 including further: upon the circumstance that said person does not have access to said recording medium, then said sufferer conducts a later session of his thoughts and feelings at a time when he is available for said recording medium.

22. The therapeutic process of claim 17 including further: a professional practitioner, including but not limited to a psychologist and a therapist, assists said person in how to conduct a session.

23. The therapeutic process of claim 17 including further: a professional practitioner, including but not limited to a psychologist and a therapist, conducts said therapeutic process in an institutional environment.

24. The therapeutic process of claim 17 including further: a professional practitioner, including but not limited to a psychologist and a therapist, assists a person in formulating encouragingly verbal messages for said person to conduct a session.

25. The therapeutic process of claim 17 including further: a professional practitioner, including but not limited to a psychologist and a therapist, assists a person in formulating verbally therapeutic messages for said person to conduct in a sessions.

26. The therapeutic process of claim 17 including further: said process is taught within a particular venue; said venue includes, but not limited to, the respective venues of lecture appearances, seminars, workshops, colleges and schools, businesses, work place environments, military institutions, community workshops, governmental institutions, health-care institutions, mental health institutions, prisons, and reformatories.

27. The therapeutic process of claim 17 including further: a description of said process is broadcasted by a broadcast station; and said station includes, but not limited to, respective television stations, radio stations, and computer-based broadcasts.

29. The therapeutic process of claim 17 including further: a description of said process is published in a publication; and said publication includes, but not limited to, respective text publications and computer-based publications.

30. The therapeutic process of claim 17 including further: said process is conducted within an institution; and said institution includes, but not limited to, respective lecture appearances, seminars, workshops, colleges and schools, churches, businesses, churches, work place environments, military establishments, community workshops, governmental establishments, health-care establishments, mental health establishments, prisons, and reformatories.

31. The therapeutic process of claim 17 including further: said recording medium having a recording unit; and a choice of recording units being, but not limited to, an audio cassette tape, a writable digitized disc, and a computerized recording program; and upon said recording unit not being fully recorded after completing a session, then said person records a successive session onto said recording unit.

32. The therapeutic process of claim 31 including further: said person alternates multiple recording units between said sessions and the listening of said recorded sessions; said person commences to record new sessions over prior recorded sessions on an existing recording unit after another recording unit has been completed with the most recent recorded sessions of said person.

33. The therapeutic process of claim 17 including further: said recording medium having a recording unit; and a choice of recording units being, but not limited to, an audio cassette tape, a writable digitized disc, and a computerized recording program; and said sufferer plays respective peripheral sounds including, but not limited to, music and ocean waves, for playing in the background of said session so as to be recorded onto said recording unit of the ensuing said recorded session.

34. The therapeutic process of claim 17 including further: while recording a session, point in time information is recorded onto the respective recorded session as to when said session is recorded; a choice of data for said information includes a day of week, a calendar date, and a time of day.

35. A therapeutic process, called inflectional therapy, for diminishing the mental ailments of depression and anxiety within an institution, and said process comprising a session; and said session consist of a sufferer of a ailment of said ailments recording [her]/his thoughts and feelings of said ailment by verbal expression onto a recording medium during the actual ailment and thus producing a recorded session of said session; a choice of recording mediums for said session includes audio tape recording, video recording with audio recording, audio recording by electronic digitalization, and recording by writing; and an administrator of said institution providing said sufferer with a private area for conducting said session; and said process including another session as needed by said sufferer; and said sufferer listening to said recorded session as needed for examining his thoughts and feelings; and said process having the therapeutic value of said sufferer expressing his thoughts and feelings in a session; and said process having the resulting therapeutic value of said sufferer externally examining his thoughts and feelings by listening to a recorded session; and the recorded session being personal of said sufferer by having recorded the details of private information of said sufferer's thoughts and feelings.

36. The therapeutic process of claim 35 including further: said institution providing a specified time period for conducting said session.

37. The therapeutic process of claim 35 including further: said recorded session being retained by said sufferer.

38. The therapeutic process of claim 35 including further: said process is taught within a particular venue; said venue includes, but not limited to, the respective venues of lecture appearances, seminars, workshops, colleges and schools,, church, businesses, work place environments, military institutions, community workshops, governmental institutions, health-care institutions, mental health institutions, prisons, and reformatories.

39. The therapeutic process of claim 35 including further: a description of said process is broadcasted by a broadcast station; and said station includes, but not limited to, respective television stations, radio stations, and computer-based broadcasts.

40. The therapeutic process of claim 35 including further: a description of said process is published in a publication; and said publication includes, but not limited to, respective text publications and computer-based publications.

41. The therapeutic process of claim 35 including further: said process is conducted within an institution; and said institution includes, but not limited to, respective lecture appearances, seminars, workshops, colleges and schools, churches, businesses, churches, work place environments, military establishments, community workshops, governmental establishments, health-care establishments, mental health establishments, prisons, and reformatories.

42. The therapeutic process of claim 35 including further: an agent of said institution, including but not limited to a psychologist and a therapist, assist said sufferer in how to conduct a session.

43. The therapeutic process of claim 35 including further: while recording a session, point in time information is recorded onto the respective recorded session as to when said session is recorded; a choice of data for said information includes a day of week, a calendar date, and a time of day.
